(19) United States
(12) Patent Application Publication
(10) Pub. No.: US 2026/0112489 A1
(43) Pub. Date: Apr. 23, 2026

(54) SYSTEM

(71) Applicant: SOFTBANK GROUP CORP., Tokyo (JP)

(72) Inventor: Mitsuhiro OGAWA, Tokyo (JP)

(73) Assignee: SOFTBANK GROUP CORP., Tokyo (JP)

(21) Appl. No.: 19/357,189

(22) Filed: Oct. 14, 2025

(30) Foreign Application Priority Data
Oct. 17, 2024 (JP) 2024-181764

(51) Int. Cl. G16H 50/20; G16H 50/70

(52) U.S. Cl. CPC G16H 50/20; G16H 50/70

(57) ABSTRACT

A system includes a processor that inputs diagnostic information and image data of a patient, searches for similar cases from a past medical case database based on the data input through the input means, generates a plurality of treatment plans based on the result obtained from the searching, calculates a suitability rate for the generated treatment options, and visualizes and presents evaluation results obtained by the suitability calculation.

### CROSS-REFERENCE TO RELATED APPLICATION

This application is based on and claims priority under 35 USC 119 from Japanese Patent Application No. 2024-181764 filed on October 17, 2024 the disclosure of which is incorporated by reference herein.

### BACKGROUND

### TECHNICAL FIELD

The present disclosure relates to a system.

### RELATED ART

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

In conventional clinical environments, obtaining a second opinion for patients with cancer or serious diseases often requires significant time and effort, as patients and their families must individually seek expert advice and treatment options. Additionally, it is typically challenging for patients or their families to comprehensively compare treatment options, evaluate their suitability, access relevant medical institutions, and make informed decisions in a timely manner. As a result, there is a need for an efficient and user-friendly system that can analyze patient information, propose appropriate treatment options, and provide reliable comparison and access to medical institutions.

### SUMMARY

To address these issues, the present invention provides a system comprising a processor configured to input diagnostic information and image data of a patient, search a past medical case database for similar cases, generate a plurality of treatment plans based on the analysis, calculate suitability rates for each treatment option, and visualize the evaluation results for user presentation. The processor may also select related medical institutions for each treatment plan and provide their location and contact information, as well as display comparative treatment information in graph or table format. This system enables patients and their families to quickly and accurately obtain second opinions, compare treatment strategies, and select optimal medical institutions for consultation and therapy.

“diagnostic information” means medical information related to the identification and assessment of a patient’s disease or health condition, including but not limited to clinical findings, laboratory results, and descriptions of symptoms.

“image data” means digital representations of medical images acquired through modalities such as CT, MRI, X-ray, or ultrasound, used for diagnosing and evaluating the health status of a patient.

“input means” means a component or interface of the system configured to receive and record diagnostic information and image data of a patient, such as a graphical user interface, data entry field, or image upload function.

“processor” means a hardware or software processing unit within the system capable of executing program instructions for analyzing data, generating options, and performing evaluations.

“past medical case database” means a collection of stored records of previous patient cases, including diagnostic information, image data, treatment histories, and outcomes.

“similar cases” means past patient cases in the database whose diagnostic information and/or image data correspond or closely resemble those of the current patient.

“treatment plan” means a proposed regimen or course of action for medical care, including specific therapies, medications, or procedures to be applied to a patient for the purpose of treating their condition.

“suitability rate” means a quantitative or qualitative measure indicating how well a proposed treatment option matches the individual circumstances and condition of the patient, often calculated based on success rate, risk, and other clinical factors.

“visualize” means to convert analysis and evaluation results into graphical, tabular, or other user-friendly formats for intuitive comprehension.

“medical institution” means a hospital, clinic, or other healthcare facility licensed to provide diagnosis, treatment, and medical services to patients.

“location information” means data specifying the geographical position or address of a medical institution.

“contact information” means details necessary for communication with a medical institution, such as telephone number, email address, or website URL.

“comparison information” means data or representations enabling the user to assess and contrast different treatment plans with respect to their success rates, risks, advantages, disadvantages, and related institution services.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### FIRST EXEMPLARY EMBODIMENT

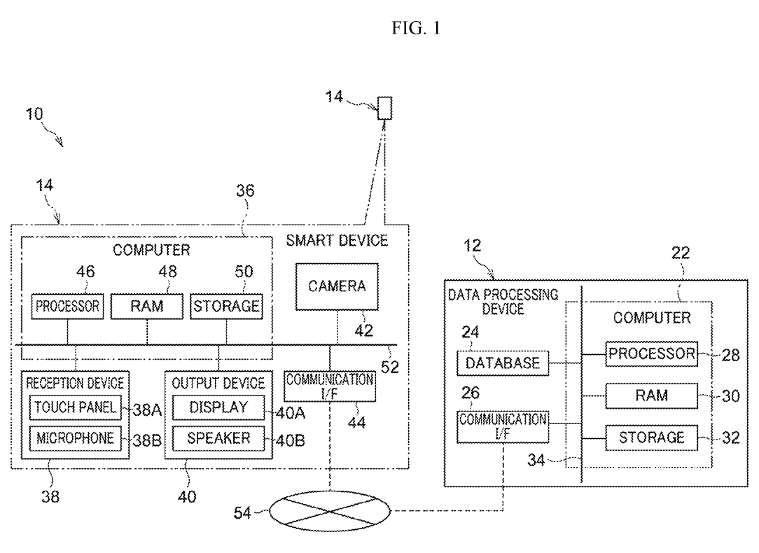

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

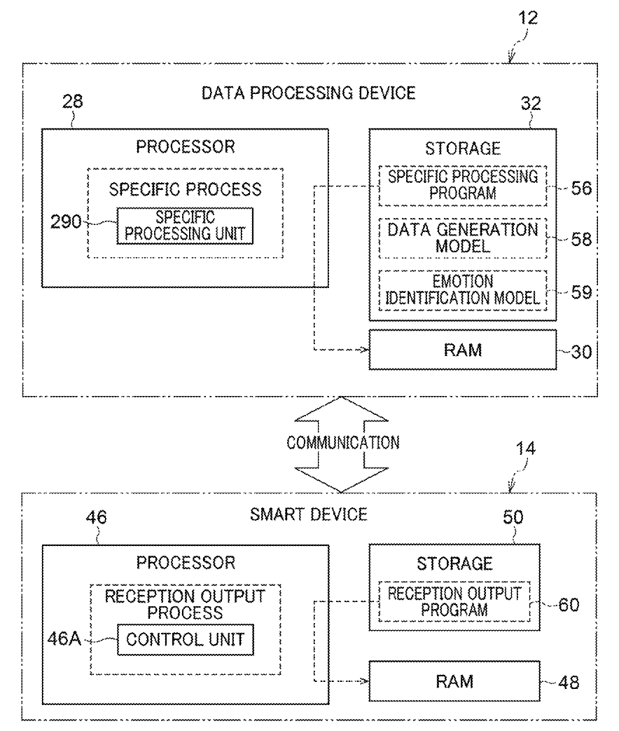

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Currently, patients seeking second opinions or optimal treatment strategies often face obstacles due to the difficulty of accurately analyzing diagnostic information and medical images, integrating up-to-date medical guidelines and historical case data, and efficiently comparing multiple treatment options. The lack of a cohesive system that validates input data, leverages generative artificial intelligence, and presents treatment proposals together with relevant risk, success, and facility information impedes timely and informed decision-making by patients and healthcare providers.

The specific processing by the specific processing unit 290 of the data processing device 12 in Example 1 is realized by the following means.

The present invention provides a server comprising a processor configured to receive diagnostic and image information, validate incoming data, analyze a case database to search for similar historical cases, utilize generative artificial intelligence to generate personalized treatment option information using prompt sentences, evaluate the compatibility of each treatment option based on statistical analysis, and present compatibility, risk, outcome, and facility information for each treatment option in a visually accessible way. This enables users to rapidly and accurately compare multiple treatment possibilities, taking into account not only clinical guidelines and previous case outcomes, but also the feasibility and accessibility of associated healthcare facilities, thus supporting improved, timely, and informed treatment decisions.

The term “diagnostic information” refers to data concerning the status, characteristics, or evaluation results of a subject’s medical condition, including but not limited to medical findings, test results, patient symptoms, and clinical assessments.

The term “image information” refers to graphical or pictorial representations obtained from diagnostic equipment, such as medical imaging modalities including computed tomography (CT), magnetic resonance imaging (MRI), ultrasound, or X-ray images, which are used to visually assess the condition of the subject.

The term “integrity and correctness validation” refers to the process of confirming that the received information is complete, formatted appropriately, free from errors, and consistent with expected input standards necessary for accurate further processing.

The term “case database” refers to a structured data repository storing historical case information, including medical conditions, diagnostic data, images, treatments performed, outcomes, and associated metadata.

The term “similar case information” refers to historical case records in the case database that are identified as having comparable diagnostic profiles, image characteristics, and relevant attributes to the currently processed subject.

The term “guideline information” refers to standardized sets of recommended medical practices or treatment protocols issued by recognized healthcare or professional organizations and intended to inform and guide treatment decisions.

The term “generative information processing apparatus” refers to an information processing system or module implementing a generative artificial intelligence model capable of generating new content, such as treatment option proposals, based on provided prompt sentences and input data.

The term “prompt sentence” refers to a structured input query or instruction supplied to the generative information processing apparatus to invoke the generation of specific informational outputs, such as treatment options.

The term “treatment option information” refers to proposed candidate methods, therapies, or medical procedures considered for addressing a subject’s medical condition, generated based on analysis and artificial intelligence processing.

The term “statistical information” refers to quantitative or qualitative data, such as risk rates, success probabilities, demographic factors, and outcome summaries, used to evaluate and compare the appropriateness of each treatment option.

The term “compatibility score” refers to a calculated indicator, derived from statistical or computational analysis, that expresses the degree of matching or suitability of a particular treatment option for a given subject.

The term “risk information” refers to data describing potential adverse effects, complications, probabilities of failure, and other hazards associated with proposed treatment options.

The term “outcome information” refers to results or consequences previously observed in similar medical cases, such as recovery rates, side effects, long-term health status, or other measures of treatment effectiveness.

The term “facility information” refers to data concerning healthcare establishments capable of providing the proposed treatment options, including but not limited to their location data and means of contact.

The term “visually accessible manner” refers to a format or method suitable for display on a user interface, such as graphical presentations, charts, or tables, that facilitate rapid and accurate understanding by a user.

One embodiment of the invention provides an information processing system comprising a processor, a terminal device, and user interface functionality, capable of receiving, validating, analyzing, generating, and displaying treatment option information based on subject-specific medical data.

The terminal can be implemented as any general-purpose computing device, such as a personal computer, tablet, or smartphone equipped with a web browser or a dedicated client application. The user operates the terminal to input subject diagnostic information, such as personal details, clinical findings, and medical image information including CT, MRI, or X-ray images. The terminal executes data integrity and validation routines using software such as JavaScript, Python, or built-in form validation functions to confirm the completeness and correctness of the data before transmission.

The server includes a processor and storage device, and can be realized as a general-purpose server computer running standard operating systems, for example, a Linux-based server equipped with Python, Django, and machine learning libraries such as TensorFlow or scikit-learn. The server receives the subject’s information from the terminal over a secure data transmission protocol such as HTTPS, and then conducts a comprehensive validation check of the incoming data using backend validation software scripts.

Upon successful validation, the server executes a data analysis process. The processor accesses a case database, which can be a structured relational database (for example, PostgreSQL), to retrieve historical case information. The server compares the input subject data with stored cases using pattern recognition algorithms, such as k-nearest neighbors or clustering algorithms implemented in Python or scikit-learn, to identify similar cases.

Based on the extracted similar case information and the latest guideline information obtained from publicly available medical standards, the server automatically generates a prompt sentence. The server then interfaces with a generative artificial intelligence model (for example, a model conforming to the transformer architecture, such as GPT-4 or an equivalent large language model running locally or through a secure API) and presents the prompt sentence to the model. The generative AI model receives the prompt and related data and outputs a set of proposed treatment options, each with suggested success rates and risk information.

The server subsequently evaluates each generated treatment option. This evaluation is done by referencing statistical information such as risk rates and historical outcome data available in the server’s information repositories. The processor computes compatibility scores indicating the fit of each treatment option to the subject’s profile using software such as NumPy and Pandas.

The server then generates a visually accessible output containing the comparative information for each treatment option. Visualization software libraries, including Matplotlib, Plotly, or D3.js, can be used to create intuitive charts or tables. The result package includes each treatment’s compatibility score, risk profile, historical outcomes, and facility information, such as locations and contact means, suitable for carrying out the treatment. These results are relayed back to the terminal, where they are rendered for the user.

As a concrete example, a user inputs information for a subject with a suspected lung tumor, including CT scan images and relevant clinical history. The terminal validates and sends this information to the server. The server identifies similar cases of lung cancer in its database, prepares the following prompt sentence for the generative AI model:

“Based on this subject’s diagnostic information and imaging data, propose both standard and new treatment options for lung cancer, and for each option, evaluate success rate and risk considering similar past cases.”

The model generates options including standard chemotherapy and advanced immunotherapy, with associated success rates and risk descriptions. The server calculates compatibility scores for each option using outcome statistics and displays the summarized results, with graphs and tables and addresses of suitable treatment facilities, on the terminal. The user can readily view the proposed therapies and supporting information in a visually accessible format.

This embodiment ensures the rapid, accurate, and user-friendly presentation of treatment option information generated and evaluated with AI techniques and delivered through well-defined validation, analysis, and visualization processes.

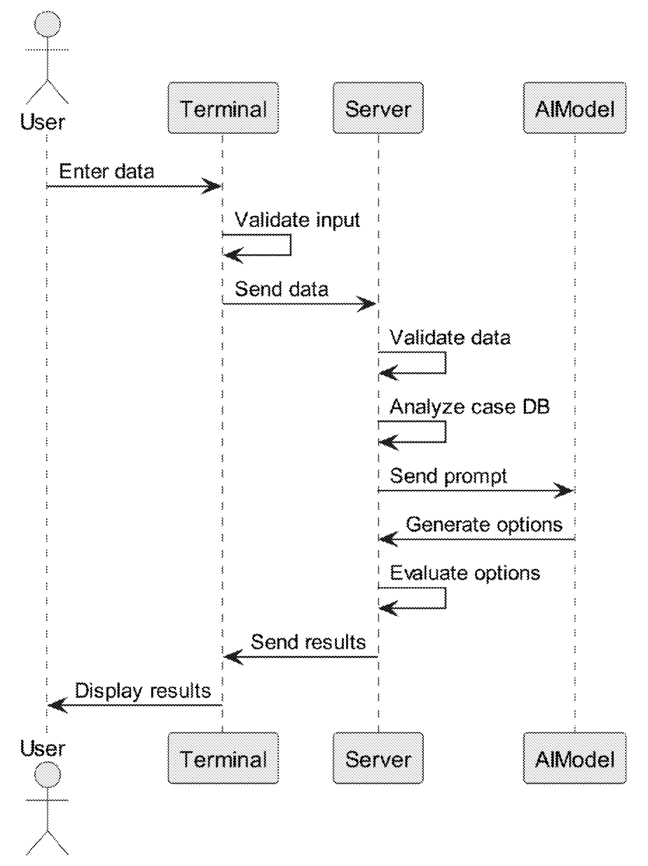

The following describes the processing flow using FIG. 11.

### Step 1

The user operates the terminal to input subject diagnostic information and medical image information, such as patient demographics, clinical history, and CT or MRI scan files, into a web application interface. The input consists of both textual and image data. The output is a completed submission form ready for validation.

### Step 2

The terminal performs local validation of the input data by checking for required fields, verifying that the image file is in an acceptable format (such as DICOM), and confirming data completeness and correctness. The input for this step is the data entered by the user. The output is validated data and an error message if any issue is detected, or a prepared data package if validation passes.

### Step 3

The terminal sends the validated data package to the server over a secure HTTPS connection. The input is the bundled and validated subject data. The output is an HTTP request containing the data, along with a server acknowledgment or error message as a response.

### Step 4

The server receives the data package and executes a backend validation process using Python scripts to ensure conformity with data standards, correctness of the image file, and completeness of all required information. The input is the uploaded data package from the terminal. The output is either a processing-ready dataset or an error response to the terminal if the data fails validation.

### Step 5

The server analyzes the validated data by accessing the case database, implemented with a structured database such as PostgreSQL. The server applies pattern recognition algorithms, such as k-nearest neighbors or clustering, using libraries like scikit-learn, to search for similar historical cases. The input is the current subject data, and the output is a list of similar reference cases along with their associated treatments and outcomes.

### Step 6

The server generates a prompt sentence that includes the subject’s diagnostic information and the list of similar cases. As an example, the prompt might be: “Based on this subject’s diagnostic information and imaging data, propose both standard and new treatment options for lung cancer, and for each option, evaluate success rate and risk considering similar past cases.” The input is the subject data and similar case information. The output is a prepared prompt sentence.

### Step 7

The server submits the prompt sentence to the generative AI model via a secured API call, providing the prompt and any necessary guideline data. The generative AI model processes the prompt and outputs a set of treatment option information, each with predicted success rates and risk details. The input is the prompt sentence and guideline data; the output is a set of structured treatment option proposals.

### Step 8

The server evaluates each treatment option by referencing statistical information, outcome data from the database, and relevant risk parameters. The server uses Python with libraries such as NumPy and Pandas to calculate a compatibility score for each option, indicating its suitability for the current subject. The input is the generated treatment options, together with statistical and historical case data. The output is an updated list of treatment options, each with a compatibility score and associated risk information.

### Step 9

The server visualizes the evaluated treatment options using visualization libraries such as Matplotlib or Plotly, generating bar graphs, tables, and summary charts. The input consists of the compatibility scores, risk information, outcome data, and facility information. The output is a visualization package that combines graphical and tabular representations suitable for end-user interpretation.

### Step 10

The server delivers the visualization package to the terminal through a secure response. The terminal receives and renders the graphs, tables, and facility data within its user interface. The input for this step is the visualization data sent by the server. The output is an interactive display, enabling the user to review, compare, and select among proposed treatment options based on clear, organized information.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

In modern medical practice, obtaining a rapid and reliable second opinion for patients is critically important, especially in cases of serious diseases such as cancer. However, challenges remain regarding the secure handling and processing of sensitive diagnostic data, the effective and user-friendly presentation of multiple treatment options, and the personalization of medical information based on the emotional state of the user. Existing systems often lack robust mechanisms for integrating advanced data encryption, AI-driven case analysis, comprehensive risk assessment with visualization, and adaptive personalization responsive to patient emotions, all within a single platform.

The specific processing by the specific processing unit 290 of the data processing device 12 in Application Example 1 is realized by the following means.

The present invention provides a server comprising a processor configured to acquire biological information and diagnostic image data, verify and encrypt input data, analyze the data using historical case information, generate candidate medical procedures by means of generative AI technology, calculate suitability and risk for each candidate, visualize comparative results in graphical or tabular format, estimate the emotional state of the user and adapt information presentation accordingly, and manage data exchange among all functional units. This enables secure, intelligent, and personalized support for second opinion decision-making by patients and healthcare providers, while ensuring data security, comprehensible multi-option treatment guidance, and user-centric interaction based on emotional status.

The term “biological information” refers to data representing the physical or physiological characteristics of a subject, including but not limited to age, sex, medical history, and clinical examination findings.

The term “diagnostic image data” refers to electronic data generated by imaging devices, such as computed tomography (CT) or magnetic resonance imaging (MRI) machines, that visually depict anatomical or pathological conditions for clinical purposes.

The term “information acquisition unit” refers to a hardware or software component that obtains and receives biological information and diagnostic image data from users or external devices.

The term “information processing unit” refers to a hardware or software component that processes the acquired data, including operations such as verifying integrity, encrypting information, and transmitting the data to other system components.

The term “external processing apparatus” refers to any computing device or networked system, remote from the information acquisition unit, that performs storage, analysis, or further processing of the transmitted data.

The term “information analysis unit” refers to a hardware or software component that retrieves and compares the input data with a repository of historical case information, using methods such as pattern recognition or machine learning.

The term “information storage device” refers to a data repository, such as a database or memory unit, that stores historical diagnostic case records, medical knowledge, and other reference data used in analysis and decision support.

The term “information generation unit” refers to a hardware or software component that automatically creates candidate medical procedures or treatment options using input data and analysis results, potentially with the aid of generative artificial intelligence models.

The term “medical procedure candidate” refers to a proposed course of diagnosis or treatment, generated as an option based on analysis of the subject’s data and existing medical knowledge.

The term “information evaluation unit” refers to a hardware or software component that determines the appropriateness, effectiveness, and associated risks of each candidate medical procedure using statistical algorithms and stored reference criteria.

The term “predefined criteria” refers to reference parameters or rules, such as clinical guidelines, outcome statistics, or risk factors, used to assess the suitability and risk of each candidate medical procedure.

The term “information visualization unit” refers to a hardware or software component that formats evaluation outcomes and comparative data into graphical or tabular representations for output to a user.

The term “graphical data” refers to data presented in a graphical format, such as charts, graphs, or diagrams, that visually communicate quantitative or comparative treatment evaluation results.

The term “tabular data” refers to data presented in table format, organizing information into columns and rows for easy comparison of treatment options.

The term “emotion estimation unit” refers to a hardware or software component that determines the psychological or emotional state of a user by analyzing textual entries or audio data.

The term “processing control unit” refers to a hardware or software component responsible for orchestrating and managing the flow of data, tasks, and instructions among multiple functional units of the system.

The term “coordinate information” refers to spatial data, such as latitude and longitude, that specifies the geographical location of an external medical service provider.

The term “contact information” refers to data enabling communication with an external medical service provider, including telephone numbers, email addresses, or web access information.

The term “external medical service provider” refers to an entity, such as a hospital or clinic, that is independent from the user and system, and that delivers diagnostic or therapeutic services.

An embodiment of the invention will be described in detail below, based on the aforementioned claims. The present invention is implemented as a system comprising a server, a terminal, and a user interface, where each functional unit operates in a coordinated manner to enable secure, intelligent, and personalized support for medical second opinions.

The server is constituted by a processor and data storage apparatus such as a cloud server or dedicated on-premise computational device. The server incorporates hardware resources, for example, x86-based servers running an operating system such as Ubuntu Linux, and employs software platforms including Python, Django or Flask for backend development, TensorFlow or PyTorch for AI analysis and inference, and PostgreSQL or MongoDB for medical historical case storage. For data encryption, the server utilizes cryptographic libraries such as OpenSSL.

The terminal may take the form of a personal computer, tablet, or smartphone, and can implement the user interface through either a web-based application or a native app compatible with operating systems such as iOS or Android. The terminal utilizes software such as a React-based web browser frontend, and JavaScript for validation and anonymization functions.

First, the user enters biological information (such as age, sex, and medical history) and uploads diagnostic image data (including, for example, CT or MRI images) using the terminal interface. The terminal validates the completeness and integrity of the user input and images, then encrypts the data using a cryptography library (such as the Web Crypto API or OpenSSL) before transmitting it to the server via a secure communication protocol such as HTTPS with TLS.

Upon receiving and decrypting the data, the server's information analysis unit compares the new patient data against a database of past diagnostic cases using a combination of Natural Language Processing and machine learning algorithms, such as a TensorFlow-based classifier for image pattern recognition and a BERT-based model for analysis of clinical textual information. The server retrieves similar historical cases and relevant outcome data from its information storage device.

The information generation unit deployed on the server automatically outputs a list of candidate medical procedures and treatment options. This process may employ generative AI models, such as a large language model optimized for medical guideline reference and multi-option synthesis. These options include standard and novel treatment approaches, and are tailored by the AI engine based on the analyzed patient condition.

The server's information evaluation unit processes each candidate option using reference criteria drawn from the database, such as recorded clinical guidelines and statistical outcomes, and computes suitability scores and risk probabilities for each. Tools such as pandas and NumPy facilitate this analytical computation.

The information visualization unit formats the evaluation results as interactive bar charts, tables, or other visual forms using visualization libraries such as D3.js or Chart.js. The visualization style and level of detail are dynamically adjusted according to the emotional state of the user, as determined by the emotion estimation unit. This emotion estimation unit analyzes the user's textual input or, where permitted, audio input captured by the terminal. Sentiment is assessed using a fine-tuned BERT or equivalent model, and the presentation is simplified or supplemented with supportive messaging if a high level of stress or anxiety is detected.

The processing control unit orchestrates data flow between the terminal and server, ensuring that all information is synchronized and securely managed.

### An illustrative use case is as follows:

A user wishes to seek a second opinion regarding lung cancer therapy. Using a tablet device, the user uploads CT scan data and enters clinical history. The terminal validates and encrypts the data, which is securely sent to the server. The server identifies similar cases using AI, generates a list of treatment options including standard chemotherapy and new immunotherapies, estimates the probabilities of success and risk for each, and creates comparative visuals. As the system detects anxiety in the user’s free-text input, it presents the treatment summary in a simplified manner with added supportive comments and suggests local providers.

### An example of a prompt sentence for the generative AI model in this embodiment is as follows:

Please analyze the following patient data (age: 62, male, history of smoking, CT/MRI images attached) and generate treatment options for lung cancer, including success probabilities and risk factors in a user-friendly visual format. If the user appears anxious, keep explanations simple and provide supportive messages.

This embodiment enables users to quickly, securely, and accurately receive comprehensive second opinion guidance that is not only clinically relevant but also personalized according to their emotional state, thereby significantly improving the quality of patient experience and decision-making.

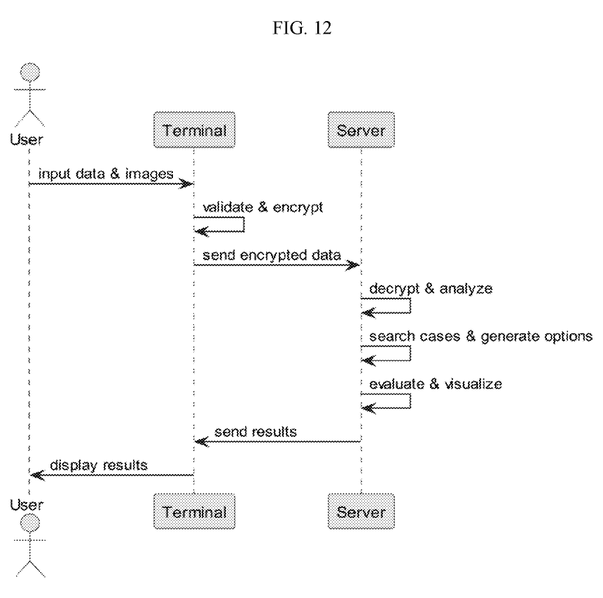

### The following describes the processing flow using FIG. 12.

### Step 1

The user inputs biological information, such as age, sex, medical history, and clinical symptoms, and uploads diagnostic image data, such as CT or MRI scans, through the terminal’s web user interface.

Input: User-entered patient details and attached medical images.

Output: Raw user data captured and temporarily stored on the terminal’s memory.

Specific action: The user fills out form fields and selects images using a browser or native app.

### Step 2

The terminal validates the input data for completeness, checks if all required fields are filled in, and verifies the file format and integrity of the uploaded images. The terminal anonymizes metadata if necessary and applies local preprocessing such as image resizing or compression.

Input: Raw user data collected in Step 1.

Output: Cleaned, validated, and optionally transformed data, ready for secure transmission.

Specific action: JavaScript routines check data completeness, file types, and image sizes.

### Step 3

The terminal encrypts the validated data using a cryptography library (such as Web Crypto API) and initiates a secure session with the server using HTTPS (TLS protocol). The encrypted data is then transmitted to the server via a secure POST request.

Input: Validated and processed patient data.

Output: Encrypted payload transmitted to the server.

Specific action: The terminal uses built-in encryption APIs, and establishes a secure HTTPS connection before sending the request.

### Step 4

The server receives the encrypted data and decrypts it using a cryptography library (such as OpenSSL). The server then parses the received information and organizes it into structured data objects for further analysis.

Input: Encrypted payload from the terminal.

Output: Decrypted and structured patient data available on the server.

Specific action: The server runs a decryption routine and reformats the payload into a database-compatible structure.

### Step 5

The server analyzes the structured patient data by comparing it with entries in the medical case database. It uses natural language processing and machine learning models, such as TensorFlow for image classification and a BERT model for disease description matching, to identify similar historical cases.

Input: Decrypted, structured patient data.

Output: A list of similar past cases along with their outcomes and treatments.

Specific action: The server executes predictive model inference and similarity searches within the database.

### Step 6

The server generates a set of candidate treatment options using a generative AI model, referencing both the current patient’s data and the outcomes of matched historical cases. Each option is described in detail, including suggested medications or procedures.

Input: List of similar cases and patient details.

Output: Multiple candidate treatment strategies, each with concise descriptions.

Specific action: The server queries the generative model and synthesizes tailored treatment recommendations.

### Step 7

The server evaluates each treatment option for suitability and risk using statistical algorithms and reference criteria, such as clinical guidelines and empirical success rates. The evaluation generates quantitative scores and risk classifications for each option.

Input: Set of candidate treatment options and analytics data.

Output: Success probabilities and risk scores assigned to each treatment option.

Specific action: The server uses tools such as pandas and NumPy to run calculations and ranking.

### Step 8

The terminal, or in some cases the server, monitors the user’s emotional state by analyzing input text or audio responses using a sentiment analysis engine, such as a fine-tuned BERT model. The emotional state is classified (e.g., calm, anxious, stressed).

Input: User interaction logs, free text, or voice data.

Output: Emotional state classification label(s) for the user.

Specific action: The system captures and analyzes user feedback in real time.

### Step 9

The server personalizes the visualization and presentation of evaluation results based on the detected emotional state. It uses a visualization library (such as D3.js) to generate bar graphs, tables, or summary messages. If the user is anxious, the presentation is simplified and supportive guidance is added.

Input: Evaluation results and emotional state classification.

Output: Customized data visualizations and accompanying text, prepared for user display.

Specific action: The server determines visualization style and output content before sending to terminal.

### Step 10

The terminal displays the personalized results and treatment options, including visual comparisons and supportive messaging if needed, using the web or app interface. The user can interact with graphs, explore options, or access additional resources such as local medical provider information.

Input: Visualized and personalized output from the server.

Output: Interactive presentation delivered to the user.

Specific action: The terminal loads and renders charts/tables, enables user clicks or taps for deeper exploration.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

In the field of personalized medical information provision, there exists a problem in that conventional systems do not sufficiently consider the emotional state of patients and often provide overwhelming, non-individualized information, leading to increased anxiety and decreased effectiveness in decision-making, particularly for patients facing serious illnesses. Furthermore, there is a lack of integration between medical data analysis, generation of treatment options, evaluation of those options, and the understanding of the emotional context of the user, resulting in non-optimal guidance and support.

The specific processing by the specific processing unit 290 of the data processing device 12 in Example 2 is realized by the following means.

The present invention provides a server comprising a processor configured to acquire biometric information and image information, analyze and compare the information with stored case data to select similar cases, generate multiple response option proposals using a knowledge-based algorithm, calculate statistical evaluation values for each proposal, determine the emotional state of the user based on voice or text input through emotion estimation logic, and adjust and control the display of information in accordance with the evaluation results and the user’s emotional state. This enables the provision of personalized, suitable, and emotionally sensitive medical options to the user, thereby reducing patient anxiety and improving the overall effectiveness of medical decision support.

The term “biometric information” refers to data relating to physical or physiological characteristics of a subject, such as age, gender, medical history, vital signs, or other health-related attributes.

The term “image information” refers to medical imaging data, including but not limited to visual representations obtained from modalities such as computed tomography (CT), magnetic resonance imaging (MRI), or other diagnostic imaging technologies.

The term “input device” refers to any hardware or software interface that enables the acquisition and entry of biometric information and image information into the system.

The term “storage device” refers to a computing resource, such as a memory or database, for the retention and retrieval of case information, medical records, and associated data.

The term “case information” refers to structured data pertaining to previous medical situations, diagnoses, treatments, outcomes, and related contextual information stored in the storage device.

The term “information analysis logic” refers to a software or hardware implemented logic that compares newly input information to stored case information and identifies relevant or similar cases.

The term “knowledge acquisition algorithm” refers to a computational procedure or model, including artificial intelligence and generative models, that generates multiple response option proposals based on analyzed information.

The term “response option proposal” refers to a possible medical solution, treatment method, or recommended action generated by the system based on the subject’s data and case analysis.

The term “evaluation computation logic” refers to the process or module configured to assess and provide statistical, probabilistic, or quantitative evaluation values for each response option proposal.

The term “communication path” refers to the means of transmitting data, including voice or text data, between the user’s device and the server, which may utilize network protocols or direct connections.

The term “emotion state estimation model” refers to an algorithm or system, including machine learning models, designed to analyze input voice or text data and determine the emotional condition of the inputter.

The term “emotion determination logic” refers to hardware or software that processes analysis results from the emotion state estimation model to classify or quantify the user’s emotional state.

The term “display control logic” refers to a software or hardware configuration for adjusting and presenting visual information, such as treatment proposals, evaluation results, and emotional support information, to the user in a user interface.

The term “facility” refers to a medical institution or healthcare provider that is relevant to a generated response option proposal.

The term “location information” refers to geographical data indicating the physical place of a facility.

The term “contact information” refers to data describing methods by which a user can communicate with or access a facility, such as telephone numbers, email addresses, or web links.

The term “visual information” refers to any output data presented to the user in a graphical or pictorial format, including graphs, charts, tables, or other user interface elements.

An embodiment of the invention will be described with reference to the system and method for providing personalized medical response options based on integrated data analysis and emotional state evaluation.

The system comprises a server equipped with a processor, at least one storage device, network communication interfaces, and necessary support hardware such as memory and graphical processing units (GPUs). The system further includes one or more user terminals that can be operated by patients, healthcare professionals, or other authorized users. The terminals are configured with input devices (such as touch screens, microphones, and image scanners) and have access to secure communication functionalities such as SSL/TLS encryption.

The user operates the terminal to input biometric information, such as patient age, gender, and medical history, together with diagnostic image information including CT images or MRI images. The terminal provides a graphical user interface for data entry and image uploading. Input validation is performed by the terminal to ensure the data adheres to specified formats, such as JSON for structured data and DICOM for medical images.

The terminal encrypts and transmits the data to the server via a secure communication path. The server receives, decrypts, and temporarily stores the data in a protected area of the storage device, for instance, using an encrypted database or file system.

The server performs information analysis by comparing the newly received data against case information stored in its storage device. The information analysis logic may employ relational database technology such as PostgreSQL, non-relational technology such as MongoDB, and search frameworks such as Elasticsearch. For image processing and feature extraction, the server may utilize image processing libraries, including OpenCV and DICOM parsers. For detection and classification of abnormal features in medical images, neural network models such as the U-Net or ResNet architectures can be implemented and executed on GPU devices.

The knowledge acquisition algorithm, which may consist of a generative artificial intelligence model (for example, a large language model trained on medical literature and case reports), receives structured data and case context as input and generates plural response option proposals. Suitable AI models include, but are not limited to, transformer-based language models and custom-trained natural language generation models.

The server evaluates the generated response options using evaluation computation logic, which may involve statistical analysis by software frameworks such as scikit-learn, TensorFlow, or similar machine learning toolkits. Each response option is assessed, and quantitative attributes such as probabilistic fit scores or outcome likelihoods are calculated.

Additionally, the server receives text or voice input from the user, which is first converted to machine-readable text via a speech-to-text engine (e.g., Google Speech-to-Text API or open source alternatives). The emotion state estimation model, built using natural language processing toolkits such as spaCy, NLTK, or emotion classification models, analyzes the input and determines the emotional state of the user along axes such as anxiety, stress, or confidence.

Based on the results of both the option evaluation and the emotion determination, the server’s display control logic customizes the presentation for the user. The server generates visual information, which may include comparative bar charts, tables, explanatory textual content, and optionally additional, reassuring or supporting information relevant to the user's detected emotional state. Visualization software such as Chart.js or D3.js can be used for this purpose. The display also includes facility information, such as locations and contacts, related to the generated response options.

The server transmits the tailored data back to the terminal, which then presents the information to the user through a graphical user interface. The user can review options, see relevant statistics, understand related facility locations, and receive emotional support messages.

For example, when a user seeks a second opinion for a suspected lung cancer case, the system receives the input data, analyzes patient and image information, compares with database cases, generates multiple treatment options, such as traditional chemotherapy and new immunotherapy approaches, assigns fit probabilities to each, and determines that the user is anxious. The system therefore adds reassuring messages about recent treatment success rates and presents nearby hospital contact info.

### An example of a prompt sentence provided to the generative AI model in this context is:

"Based on the diagnostic data input by the user, including CT/MRI images, patient demographics, and medical history, please provide the optimal second opinion treatment options. Furthermore, use the emotion engine to supply information in a way that reduces user stress and supports emotional well-being in your response."

This embodiment enables flexible adaptation to changes in available medical knowledge, as well as real-time personalization for each user situation, thus supporting high-quality, emotionally considerate medical decision-making.

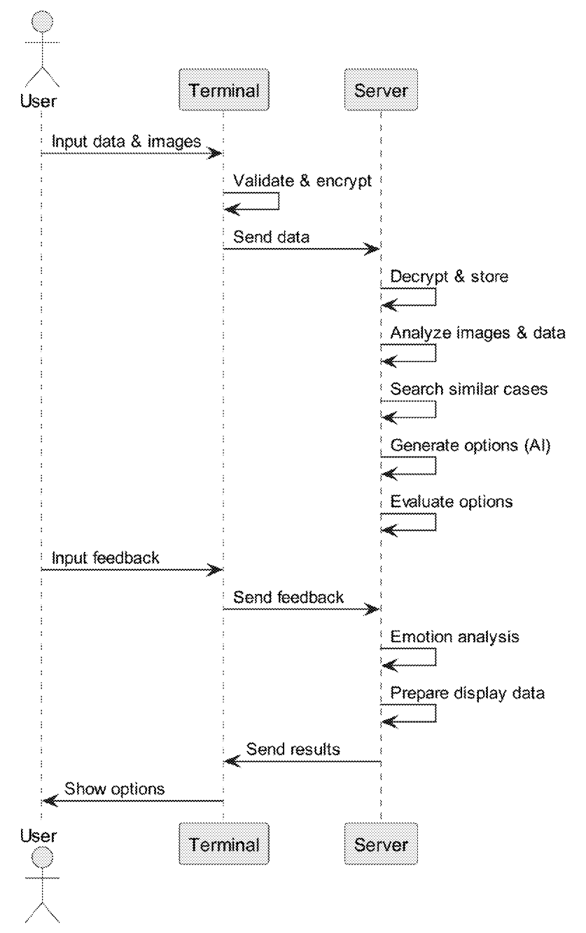

### The following describes the processing flow using FIG. 13.

### Step 1

The user enters biometric information such as age, gender, and medical history, and uploads medical images like CT or MRI scans into the terminal using a graphical user interface.

Input: Patient’s demographic data, medical history, and diagnostic images.

Data processing/output: The terminal checks the input data format for errors and completeness, and prepares the data in a unified structure for secure transmission.

### Step 2

The terminal validates the input data, converts it if necessary (e.g., from DICOM to a readable format), encrypts the unified data structure and image files using SSL/TLS encryption, and logs the event in a local audit log.

Input: Structured patient data and medical images from the user.

Data processing/output: Encrypted and validated data ready for secure transmission to the server.

### Step 3

The terminal sends the encrypted data to the server through a secure HTTPS connection and waits for an acknowledgment of successful delivery.

Input: Encrypted unified patient data and images.

Data processing/output: Secure network transmission and delivery confirmation.

### Step 4

The server receives the encrypted patient data and image files, verifies their integrity and authenticity, and then decrypts the data using the appropriate private key. The server stores the received information in a protected storage location.

Input: Encrypted patient data received via the network.

Data processing/output: Decrypted, structured patient data and images securely stored and ready for analysis.

### Step 5

The server processes the medical images using image processing software (such as OpenCV). It applies algorithms for feature extraction, such as nodule detection or lesion segmentation, often with neural network models.

Input: Decrypted CT/MRI images and related patient data.

Data processing/output: Extracted feature data and image analysis results identifying possible abnormalities.

### Step 6

The server compares the patient’s structured data and extracted image features to cases in its storage device, using information analysis logic implemented with database query engines.

Input: Structured patient data and extracted image features.

Data processing/output: A ranked list of similar case information and associated treatment outcomes.

### Step 7

The server generates a prompt sentence for the generative AI model that summarizes the patient’s situation, and sends the structured data and case context to the AI model. The generative AI model outputs multiple response option proposals, including explanations and references.

Input: Patient data, extracted features, and case comparison results.

Data processing/output: Multiple generated treatment option proposals with explanatory content.

### Step 8

The server evaluates each treatment option proposal using evaluation computation logic, such as statistical analysis or machine learning prediction models, to calculate suitability scores and relevant evaluation metrics.

Input: Response option proposals from the generative AI model.

Data processing/output: Fit score or probability value for each treatment option, compiled into a result set.

### Step 9

The server analyzes the user’s text or voice input using a speech-to-text engine (if needed) and an emotion state estimation model to determine the user’s emotional state (e.g., anxiety or confidence levels).

Input: User’s textual or voice feedback.

Data processing/output: Quantified emotional state data for the user.

### Step 10

The server customizes the information display by integrating treatment scores, emotional state, and facility information. The display control logic arranges the information in graphical or tabular formats, and includes personalized explanatory messages based on the user’s emotional status.

Input: Fit scores, emotional state data, and facility information.

Data processing/output: Visualization data and tailored content for user presentation.

### Step 11

The server encrypts and transmits the visualization data and tailored content to the terminal via a secure network connection.

Input: Visualization and content data for the user.

Data processing/output: Encrypted graphical and textual information delivered to the terminal.

### Step 12

The terminal receives and decrypts the data, then presents the treatment options, fit scores, supporting explanations, and personalized messages to the user through the graphical interface. The terminal logs user interactions with the display for system improvement.

Input: Visualization data and tailored messages from the server.

Data processing/output: Intuitive and supportive presentation of information to the user, enabling informed and emotionally considerate decision-making.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

In information provision systems, particularly in electronic transactions and decision-support services, conventional systems provide information or recommendations without considering the emotional state of the user. As a result, users who feel uncertainty or hesitation are not sufficiently reassured, which may lead to reluctance in proceeding with purchases or actions. Therefore, there remains a problem in delivering timely, adaptive, and reassuring information that is responsive to the user's emotional context.

The specific processing by the specific processing unit 290 of the data processing device 12 in Application Example 2 is realized by the following means.

The present invention provides a server comprising a processor configured to receive input information, search a case database for similar past cases, generate multiple solution options, calculate suitability rates, detect the user's emotional state, adjust the presentation of information according to both the suitability rates and the emotional state, visually present the adjusted information, and generate explanatory text using a generative artificial intelligence unit based on a predetermined prompt sentence. This enables the provision of information and options that are optimally adapted to both the objective circumstances and the user's emotional needs, thereby increasing user confidence and support during decision-making.

The term “information acquisition unit” refers to a component or function that receives and collects input information from a user, such as data related to user preferences, queries, or other relevant content.

The term “analysis unit” refers to a component or function that processes and analyzes the input information in order to search and identify similar cases from a database storing past cases or records.

The term “option generation unit” refers to a component or function that creates multiple solution options or proposals based on the results obtained from the analysis unit.

The term “evaluation unit” refers to a component or function that calculates the suitability rates or compatibility scores for each solution option by comparing input information with historical data.

The term “emotion recognition unit” refers to a component or function that detects and determines the emotional state of a user, such as anxiety, confidence, or hesitation, by processing input signals like facial expressions or voice data.

The term “information adjustment unit” refers to a component or function that modifies and customizes the content and format of information to be presented, based on the suitability rates and the detected emotional state.

The term “visualization unit” refers to a component or function that displays content or information in a visually accessible and comprehensible manner to the user, such as through a graphical user interface or visual display.

The term “generative artificial intelligence unit” refers to a component or function that utilizes a generative artificial intelligence model to automatically generate explanatory text or messages, particularly using a predetermined prompt sentence, to address the user’s emotional needs and provide reassurance.

The term “suitability rate” refers to a numerical or categorical value indicating the degree to which each solution option matches or fits the user's input information and context.

The term “prompt sentence” refers to a predefined instruction or query provided to the generative artificial intelligence unit to guide the generation of explanatory text or messages.

An embodiment of the present invention provides a system for adaptive information presentation and user support, which is particularly suited for use in electronic transactions, decision support, and similar information provision scenarios.

The server includes a processor configured to function as an information acquisition unit, analysis unit, option generation unit, evaluation unit, emotion recognition unit, information adjustment unit, visualization unit, and generative artificial intelligence unit. The server is connected via a network to one or more terminals, which may be general-purpose computing devices such as smartphones, tablets, or computers, and are operated by the user.

The information acquisition unit receives user input, which may include queries, preferences, or transactional information. This input can be provided through a graphical user interface or application on the terminal, using standard input methods such as touchscreens or keyboards. The collected information is transmitted securely to the server over the network using typical communication protocols, such as HTTPS.

The analysis unit on the server processes the input information and searches a case database for similar records. The case database may be implemented using general-purpose database management software, such as a relational database management system. The analysis unit operates using data processing algorithms, which may be implemented using software environments such as Python, combined with data analysis libraries like Pandas or NumPy.

The option generation unit then creates multiple solution options based on the analysis results. This may involve machine learning models or rule-based algorithms to propose relevant options for the user's circumstances. For example, the option generation may be implemented with a recommendation engine built on Scikit-learn or similar software.

The evaluation unit calculates suitability rates for each generated option. These suitability rates represent how well each option matches the user's input and historical case data. The calculation may use similarity measures such as cosine similarity and may incorporate machine learning models.

The emotion recognition unit detects the user's emotional state. This unit receives sensory data, such as images or audio, from the terminal. The sensory data is processed using computer vision software, for example, OpenCV, and emotion detection models, such as the Facial Emotion Recognition (FER) algorithm. Voice data may be analyzed using speech-to-text software and audio signal processing techniques.

The information adjustment unit modifies the form and content of the information to be presented, using the evaluation results and the detected emotional state. For example, if the emotion recognition unit determines that the user is anxious, the information adjustment unit increases the prominence of reassuring details, such as return policies, customer support, and positive reviews.

The visualization unit converts this adapted information into a format suitable for user display. The terminal presents the information using a graphical user interface, which may be a web-based dashboard or a native app screen, allowing for interactive presentation and user-friendly navigation.

The generative artificial intelligence unit generates explanatory text or messages. This unit operates by submitting a prompt sentence along with contextual information, such as the user’s detected emotion and the options generated, to a generative AI model. The generative AI model may be implemented as a cloud-based language model, such as GPT-4 or an equivalent system. The prompt sentence guides the text generation to address the user's emotional needs. An example of a prompt sentence is:

"If a user shows a hesitant or anxious facial expression before purchase, propose a method to provide information so that they feel reassured."

The server then receives the AI-generated explanatory text and includes it with the information presented to the user, thereby increasing the user’s confidence and supporting successful decision-making.

As a concrete example, a user may input details about a product they are interested in purchasing via a tablet application. If the device camera detects that the user appears anxious, the server highlights relevant assurance information (such as warranty policies and previous positive reviews) on the terminal interface and uses the generative AI unit to create a supportive message, which is displayed to the user in real time.

Accordingly, the present invention supports adaptive, personalized information delivery, responsive to both the user’s objective transaction context and subjective emotional state, using widely available hardware and software tools.

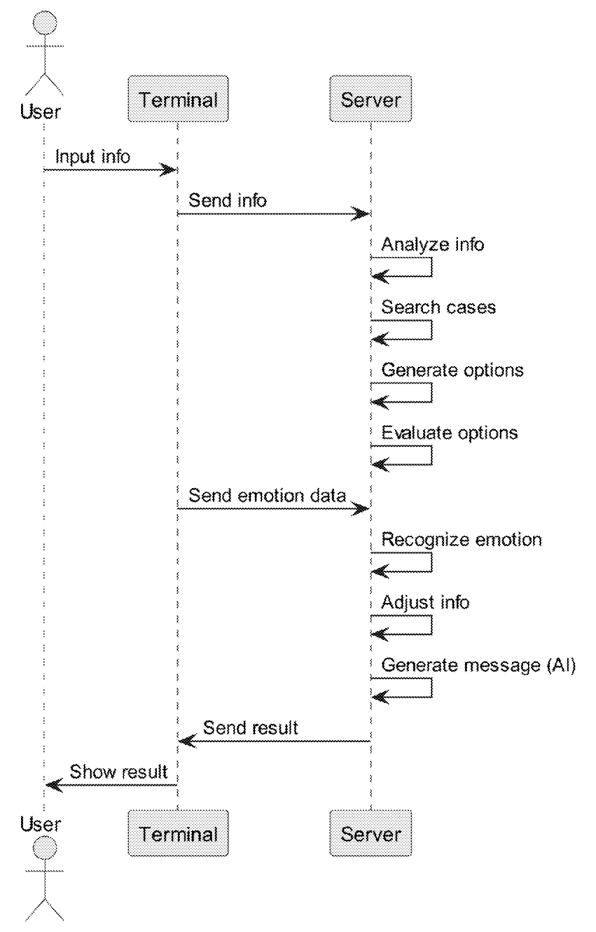

### The following describes the processing flow using FIG. 14.

### Step 1

The user inputs required information, such as product preferences and personal details, using the terminal’s graphical user interface. The terminal collects the entered data as structured input. The output of this step is a data packet containing user input information.

### Step 2

The terminal transmits the user input data packet to the server over a secure network connection. The input is the structured data from Step 1, and the output is the successful transfer of this data to the server for processing.

### Step 3

The server receives the user data and processes it with the analysis unit. Using algorithms and a case database, the server extracts keywords and searches for similar historical cases. The input is the user’s data record, and the output is a set of similar case records retrieved from the database.

### Step 4

The server uses the option generation unit to generate multiple solution options based on the results from the analysis unit. The input is the set of relevant case records, and the server applies data synthesis methods to create a list of possible options. The output is a list of solution options tailored to the user’s situation.

### Step 5

The server evaluates each generated solution option using the evaluation unit. By comparing the user’s input against characteristics of past cases, the server calculates a suitability rate for every option. The input is the solution options and case data, and the output is a list of options, each assigned a suitability score.

### Step 6

The terminal captures real-time sensory data, including facial images and voice recordings, from the user, upon user consent, using the camera and microphone. The input is user’s live expression and voice data, and the output is a set of multimedia files sent to the server.

### Step 7

The server processes the sensory data with the emotion recognition unit, employing computer vision and speech analysis algorithms to detect the user’s emotional state. The input is the image and audio files from Step 6, and the output is a classification result indicating the user’s current emotional state (e.g., anxious, neutral, confident).

### Step 8

The server adjusts the information content and layout using the information adjustment unit. Depending on the suitability scores and the detected emotional state, the server modifies what information will be emphasized or how it will be formatted. The input is the suitability rates, emotion classification, and solution options; the output is a customized information package prepared for presentation.

### Step 9

The server uses the generative artificial intelligence unit to generate a supportive explanatory message. The server constructs a prompt sentence incorporating contextual details and submits it to the generative AI model. For example, the prompt sentence could be:

"If a user shows a hesitant or anxious facial expression before purchase, propose a method to provide information so that they feel reassured."

The input is the context data and prompt, and the output is a generated textual message designed to reassure the user.

### Step 10

The server sends the entire customized information package, including the tailored solution options, suitability rates, formatted content, and the AI-generated message, to the terminal. The input is the complete set of information from previous processing steps, and the output is the rendered package transmitted to the terminal.

### Step 11

The terminal presents the received content to the user through its graphical user interface. The user sees a dynamically structured display showing product recommendations, suitability scores, reassurance information, and the AI-generated message. The input is the rendered package from the server, and the output is the visual presentation that supports and guides the user’s next action.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

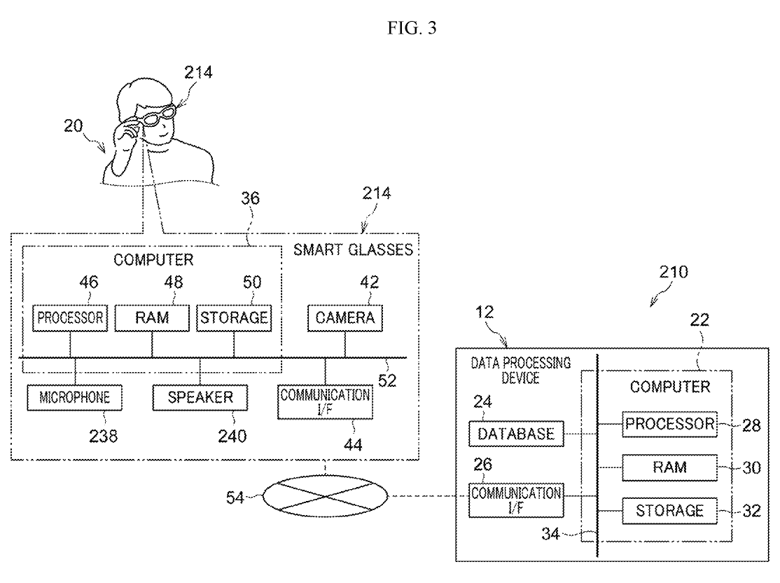

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

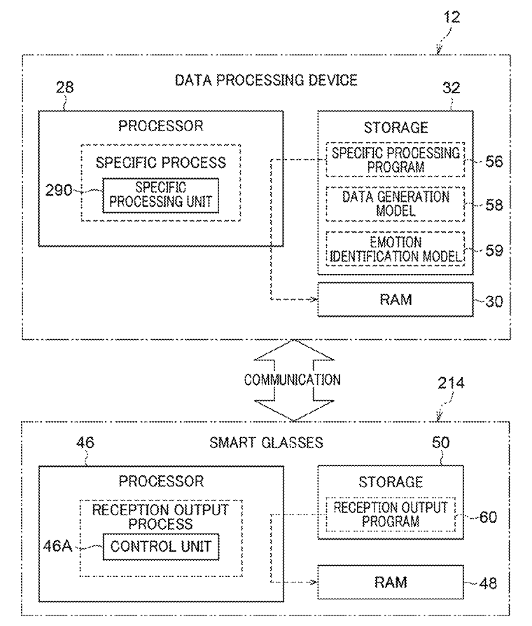

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### THIRD EXEMPLARY EMBODIMENT

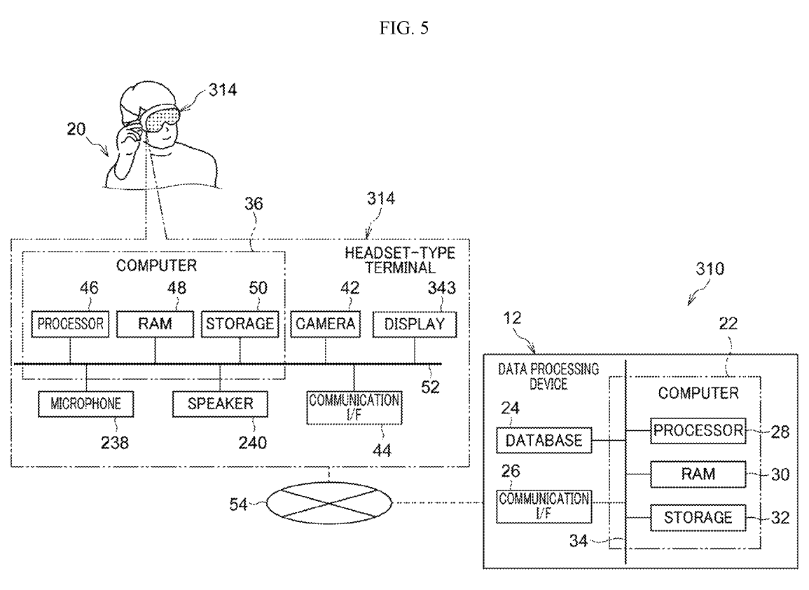

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

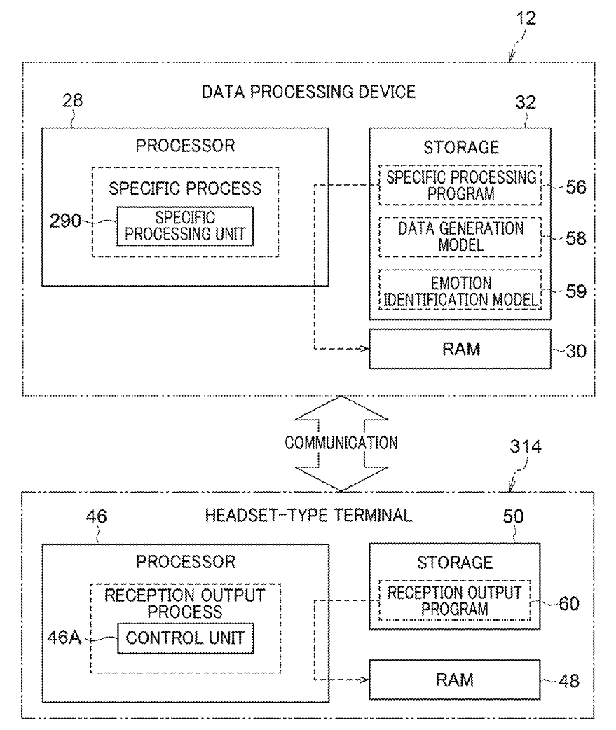

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### FOURTH EXEMPLARY EMBODIMENT

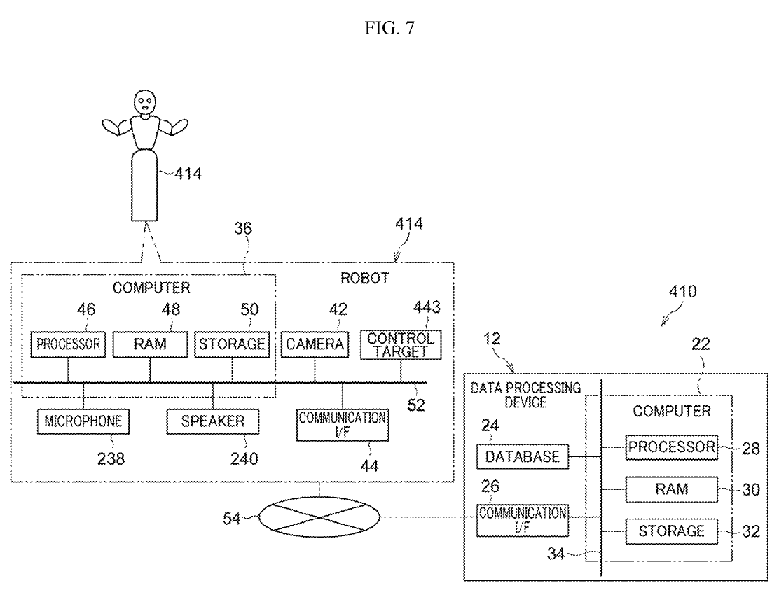

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

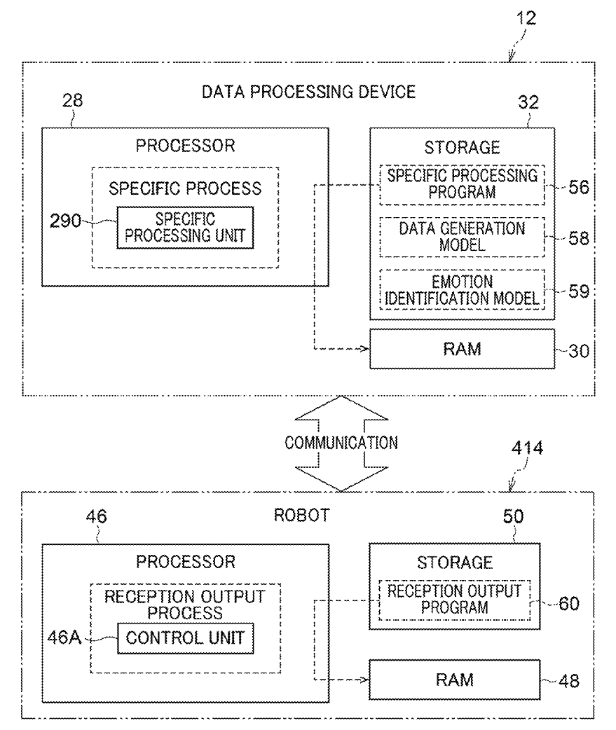

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

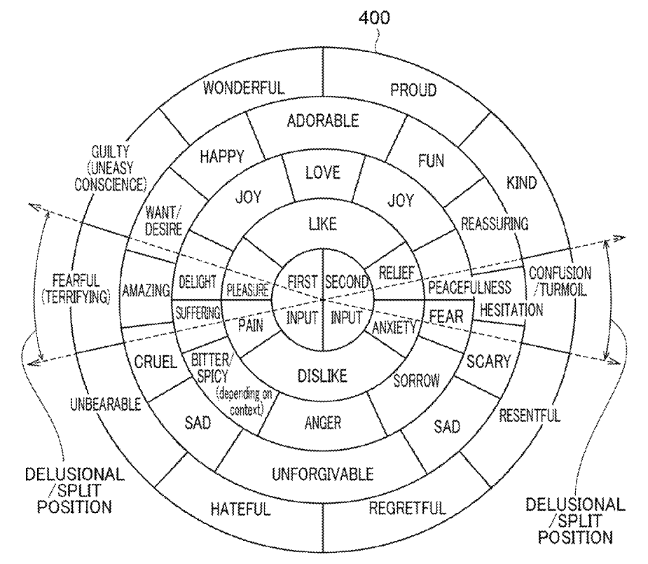

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o’clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don’t want to feel this way ever again” and “I don’t want to be chided again” is experienced in a robot. Another is a positive emotion in the area of “desire” on the reaction side. In other words, there are times when a positive feeling such as “desire more” and “want to know more” is experienced.

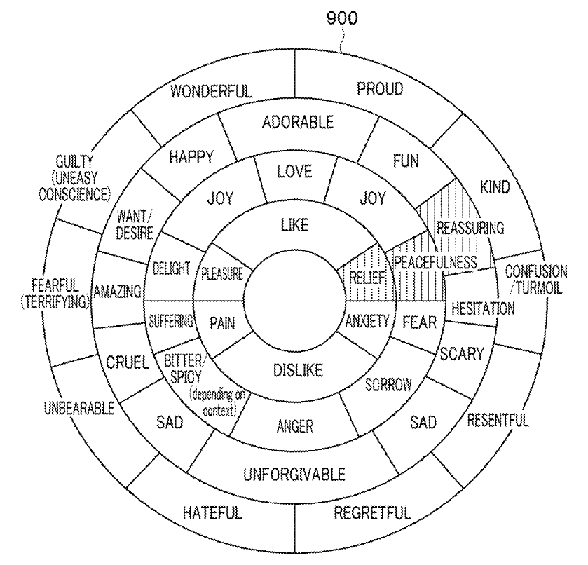

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor, wherein the processor is configured to receive subject diagnostic information and image information, validate the integrity and correctness of the received information, search for similar case information from a stored case database based on the received information, generate a prompt sentence for a generative information processing apparatus using the similar case information and latest guideline information, and obtain a set of treatment option information from the apparatus, calculate compatibility scores for the generated treatment option information based on statistical information, and display, in a visually accessible manner, compatibility information, risk information, outcome information, and facility information regarding the treatment option information.

### Supplementary 2

The system according to supplementary 1, wherein the processor is configured to display, for each treatment option information, corresponding facility information including location data and contact means.

### Supplementary 3

The system according to supplementary 1, wherein the processor is configured to present comparative information for the set of treatment option information in a graphical form or tabular form.

### Application Example 1

### Supplementary 1

A system comprising a processor, wherein the processor is configured to acquire biological information and diagnostic image data through an information acquisition unit, verify the integrity of the input data, encrypt the data, and transmit the encrypted data to an external processing apparatus through an information processing unit, analyze the transmitted data by retrieving similar historical diagnostic cases from an information storage device through an information analysis unit, generate candidate medical procedures based on the analysis results through an information generation unit, calculate suitability and risk assessments of each candidate medical procedure with reference to predefined criteria through an information evaluation unit, output the evaluation results as graphical data or tabular data through an information visualization unit, estimate a user's emotional state from input text or audio information and adjust the visualization and presentation information according to the estimated emotional state through an emotion estimation unit, and control data exchange and processing among the above units through a processing control unit.

### Supplementary 2

The system according to supplementary 1, wherein the processor is configured to present coordinate information and contact information of external medical service providers corresponding to the candidate medical procedures based on the evaluation results through the information visualization unit.

### Supplementary 3

The system according to supplementary 1, wherein the processor is configured to provide comparative information for a plurality of candidate medical procedures as graphical data or tabular data through the information visualization unit.

### Example 2

### Supplementary 1

A system comprising a processor, wherein the processor is configured to acquire biometric information and image information from an input device; compare the acquired information with case information stored in a predetermined storage device and select similar case information using information analysis logic; generate a plurality of response option proposals based on the selection result from the information analysis logic and additional relevant information by using a knowledge acquisition algorithm; calculate probability information or statistical evaluation values for each of the generated response option proposals using an evaluation computation logic; analyze input voice data or text data received through a communication path by an emotion state estimation model to determine the emotional state of an inputter; and adjust the content of information based on the information obtained from the evaluation computation logic and the emotional state determined by the emotion determination logic, and control display of the information as visual information.

### Supplementary 2

The system according to supplementary 1, wherein the processor is configured to cause the display control logic to display, together with the evaluation results of each response option proposal, information comprising location information and contact information of facilities related to each proposal.

### Supplementary 3

The system according to supplementary 1, wherein the processor is configured to cause the display control logic to present comparative information relating to the plurality of generated response option proposals in a graphical or tabular format utilizing visual elements.

### Application Example 2

Supplementary 1

A system comprising a processor, wherein the processor is configured to receive input information by an information acquisition unit, search similar cases from a past case database based on the input information by an analysis unit, generate a plurality of solution options based on a result obtained by the analysis unit by an option generation unit, calculate suitability rates of the generated solution options by an evaluation unit, detect a user emotional state by an emotion recognition unit, adjust information to be presented based on information from the evaluation unit and the emotion recognition unit by an information adjustment unit, visually present the adjusted information by a visualization unit, and generate an explanatory text that provides reassurance by a generative artificial intelligence unit.

### Supplementary 2

The system according to supplementary 1, wherein the processor is configured to generate the explanatory text using a predetermined prompt sentence based on the user emotional state and the solution options by the generative artificial intelligence unit.

### Supplementary 3

The system according to supplementary 1, wherein the processor is configured to dynamically change and display the emphasis or layout of the presented information by the visualization unit in accordance with the suitability rates and the user emotional state determined by the evaluation unit and the emotion recognition unit.

What is claimed is:

1. A system comprising a processor, wherein the processor inputs diagnostic information and image data of a patient, searches for similar cases from a past medical case database based on the data input through the input means, generates a plurality of treatment plans based on the result obtained from the searching, calculates a suitability rate for the generated treatment options, and visualizes and presents evaluation results obtained by the suitability calculation.

2. The system according to claim 1, wherein the processor selects related medical institutions for the generated treatment plans, and presents location information and contact information of the selected medical institutions.

3. The system according to claim 1, wherein the processor provides comparison information of the generated treatment plans in a graph or table format.
